(19) United States
(12) Patent Application Publication
(10) Pub. No.: US 2004/0229934 A1
(43) Pub. Date: Nov. 18, 2004

(54) Method of reducing type 2 diabetes in high risk patients

(76) Inventor: Salim Yusuf, Hamilton (CA)

Correspondence Address: EDWARDS & ANGELL, LLP, P.O. BOX 55874, BOSTON, MA, 02205, US

(21) Appl. No.: 10/783,615

(22) Filed: Feb. 20, 2004

Related U.S. Application Data
(63) Continuation of application No. 10/273,814, filed on Oct. 21, 2002.

(51) Int. Cl. A61K 31/41

(52) U.S. Cl. 514/423

(57) ABSTRACT

The present invention relates to the use of an angiotensin-converting enzyme inhibitor, such as ramipril, to reduce or prevent Type 2 diabetes in high risk patients. The present invention also relates the use of an angiotensin-converting enzyme inhibitor, such as ramipril, to reduce the decline of β-cell function, increase islet blood flow, lower aldosterone secretion, lower renal potassium wasting, increase pancreatic β-cell perfusion, reduce insulin resistance in skeletal muscles, and increase insulin-mediated glucose disposal and uptake by skeletal muscles.

### RELATED U.S. PATENT APPLICATIONS

This application for U.S. patent relates and claims priority to the following U.S. provisional application, which was filed on Oct. 17, 2001, assigned Ser. No. 60/344,495 and is entitled Method of Reducing Type 2 Diabetes in High Risk Patients, and which is incorporated in its entirety herein by reference

### U.S. PATENT APPLICATION

This application for U.S. patent is filed as a utility application under U.S.C., Title 35, §111(a).

### FIELD OF THE INVENTION

The present invention relates to the use of an angiotensin-converting enzyme inhibitor, such as ramipril, to reduce or prevent Type 2 diabetes in high risk patients by, for example, reducing the decline of β-cell function, increasing islet blood flow, lowering aldosterone secretion, lowering renal potassium wasting, increasing pancreatic β-cell perfusion, reducing insulin resistance in skeletal muscles, and increasing insulin-mediated glucose disposal and uptake by skeletal muscles.

### BACKGROUND

Type 2 diabetes is a growing clinical and public health problem. Type 2 diabetes is an important and common risk factor for the development of coronary artery disease, strokes, peripheral arterial disease, and renal and eye disease. Currently, in North America, the direct and indirect costs of diabetes and its complications exceeds $100 billion per year. This health and economic impact of diabetes is bound to increase, as the global prevalence of diabetes rises from 4.2% to 5.4% by the year 2025.

A growing amount of literature suggests that the complications of diabetes may be reduced or prevented by improving glucose control, lowering blood pressure and lipids, smoking cessation, and taking angiotensin converting enzyme (ACE) inhibitors. An even more effective approach to preventing these problems would be to prevent diabetes from developing. Whereas recent evidence from trials suggests that lifestyle modifications may reduce the risk of diabetes, the long-term adherence to such interventions has not been high. Preventive efforts related to lifestyle modification, however, are not always successful. Therefore, alternative prevention strategies that are more easily implemented, safe ad likely to prevent not only diabetes but also its chronic consequences are needed.

### SUMMARY OF THE INVENTION

The present invention overcomes and alleviates the above-mentioned drawbacks and disadvantages in the diabetes art through the discovery of a novel method to reduce or prevent Type 2 diabetes in high-risk patients.

Generally speaking, the present invention relates to a method of reducing Type 2 diabetes in patients who are at risk for developing Type 2 diabetes by administering to a patient, who is at risk for developing Type 2 diabetes, an effective amount of an angiotensin-converting enzyme inhibitor, such as ramipril, for a sufficient period of time to prevent the development of Type 2 diabetes in such patient. It is believed that the benefits of the present invention are accomplished by, for example, reducing the decline of β-cell function, increasing islet blood flow, lowering aldosterone secretion and lowering renal potassium wasting, increasing pancreatic β-cell perfusion, reducing insulin resistance in skeletal muscles, and increasing insulin-mediated glucose disposal and uptake by skeletal muscles.

Thus, the present invention is also concerned with methods of: (a) slowing or reversing the decline of β-cell function in an individual comprising administering to an individual an effective amount of an angiotensin converting enzyme inhibitor for a sufficient period of time to prevent the decline of β-cell function in such individual; (b) increasing islet blood flow in an individual comprising administering to an individual an effective amount of an angiotensin converting enzyme inhibitor for a sufficient period of time to increase islet blood flow in such individual; (c) increasing pancreatic β-cell perfusion in an individual comprising administering to an individual an effective amount of an angiotensin converting enzyme inhibitor for a sufficient period of time to increase pancreatic β-cell perfusion in such individual; (d) lowering aldosterone secretion and renal potassium wasting in an individual comprising administering to such an individual an effective amount of an angiotensin converting enzyme inhibitor for a sufficient period of time to lower aldosterone secretion and renal potassium wasting in such individual; (e) reducing insulin resistance in skeletal muscles; and (f) increasing insulin-mediated glucose disposal; and increasing insulin-mediated uptake by skeletal muscles.

While it should be appreciated by those versed in this art that any effective angiotensin converting enzyme inhibitor and any effective dosage regimen are contemplated by the present invention, ramipril is the preferred angiotensin converting enzyme inhibitor for use in accordance with the novel methods of the present invention in a dosage regimen of up to about 10 mg/day.

These and other objects, features, and advantages of the present invention may be better understood and appreciated from the following detailed description of the embodiments thereof, selected for purposes of illustration and shown in the accompanying Figures and Example. It should therefore be understood that the particular embodiments illustrating the present invention are exemplary only and not to be regarded as limitations of the present invention.

### BRIEF DESCRIPTION OF THE FIGS.

The foregoing and other objects, advantages and features of the invention, and the manner in which the same are accomplished, will become more readily apparent upon consideration of the following detailed description of the invention taken in conjunction with the accompanying Figs., wherein:

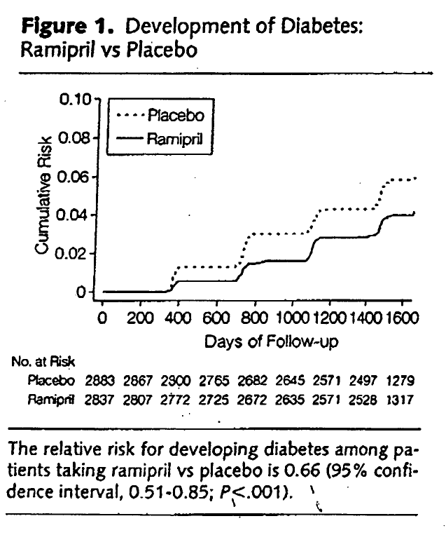

FIG. 1 illustrates the development of Diabetes Mellitus in a population treated with ramipril or placebo; and

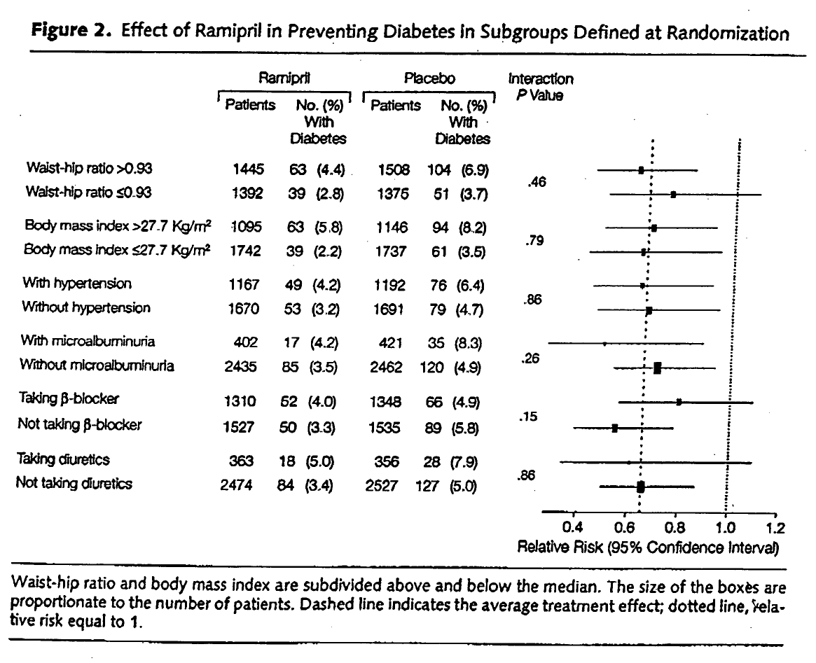

FIG. 2 illustrates results among subgroups of patients with different risk factors for developing diabetes.

### DETAILED DESCRIPTION

By way of illustrating and providing a more complete appreciation of the present invention and many of the attendant advantages thereof, the following detailed description is given concerning the novel methods for preventing Type 2 Diabetes in high risk patients.

As discussed, ramipril is associated with lower rates of new diagnosis of diabetes in high-risk individuals without known diabetes at randomization.

The following Example is given to further illustrate the present invention and is not to be considered as a limitation of this invention or any apparent variation of which are possible without departing from the spirit or scope thereof.

### EXAMPLE

The design of the Heart Outcomes Prevention Evaluation (HOPE) trial has been described in detail in previous publications. Briefly, individuals who were 55 years or older with no evidence of left ventricular dysfunction or heart failure and who had evidence of vascular disease or who had diabetes and 1 other risk factor were eligible as long as they had no indication or contraindication to receiving an ACE-inhibitor. The study was conducted in 26 hospitals in 19 countries from 1994 to 1999. All patients provided written informed consent.

Of 10576 eligible patients who participated in a run-in period during which they received 2.5 mg ramipril once daily for 1 week followed by matching placebo for 10 to 14 days, 1035 (9.8%) were excluded from randomization (3.2% for side effects, 3.7% for lack of consent). Of the remaining 9541 patients, 3654 (38.3%) had a clinical diagnosis and 5887 (61.7%) did not at randomization. This Example focuses primarily on the latter group of patients. Of these patients, 5720 were randomized to receive up to 10 mg of ramipril once per day or equivalent placebo. One hundred sixty-seven patients who were randomized to receive a low dose (2.5 mg/day) of ramipril as part of the Study to Evaluate Carotid Ultrasound changes with Ramipril and Vitamin E (SECURE). All randomized patients were also randomized to receive 400 IU of Vitamin E or placebo.

Follow-up visits occurred at 1 month and 6 months after randomization and then every 6 months (mean follow-up of 4.5 years). At each visit, we documented whether the diagnosis of diabetes had been made since the last visit.

The primary outcome of this analysis is a new diagnosis of diabetes recorded on the basis of self-report. This diagnoses was made blinded to treatment allocation and, hence, is believed likely to be unbiased. Hemoglobin A_{1c} (HbA_{1c}) levels and medications used among those diagnosed as having diabetes were also recorded. The HbA_{1c}, levels were determined locally. Values higher than 110% of the upper limit of normal for each laboratory were considered to be biochemical confirmation of diabetes.

Survival curves utilizing the Kaplan Meier and log-rank procedures were used to describe and compare the results in the 2 treatment groups. Because of the factorial design, all analyses were stratified for randomization to vitamin E or placebo. Subgroup analyses were conducted using tests of interaction in the Cox regression model.

The baseline characteristics of the patients who did not have diabetes are provided in Table 1. The proportion of patients taking study ramipril or open label ACE-inhibitors in the active group was 98.3% at 2 years and 89.7% at 4 years. The proportion taking open label ACE-inhibitors in the control group was 11.6% and 27.4% respectively.

TABLE 1
Baseline Demographics in Patients Without Diabetes Who Entered into
HOPE

| Characteristics | Ramipril Group | | Placebo Group | |
|---|---|---|---|---|
| Total No. randomized | 2837 | | 2889 | |
| | Mean (SD) | | Mean (SD) | |
| Age | 68.3 | (6.7) | 65.9 | (6.9) |
| Blood pressure, mm HG | | | | |
| Systolic | 136.4 | (19.5) | 198.7 | (19.4) |
| Diastolic | 78.2 | (10.6) | 78.7 | (10.5) |
| Heart [ ] | 86.2 | (10.8) | 68.6 | (10.8) |
| Body Mass Index | 28.9 | (3.9) | 27.2 | (4.0) |
| | No. (%) | | No. (%) | |
| Women | 583 | (20.5) | 576 | (19.9) |
| Nonwhites | 233 | (8.21) | 239 | (8.29) |
| Waist-hip ratio | 0.93 | (0.08) | 0.9 [ ] | (0.06) |
| Coronary artery disease | 2645 | (93.2) | 2893 | (93.4) |
| Myocardial Infarction | 1784 | (62.9) | 1819 | (63.1) |
| Stable, angina | 1826 | (64.4) | 1849 | (64.1) |
| Unstable angina | 881 | (30.3) | 852 | (29.8) |
| Stroke or transient | 374 | (12.2) | 918 | (11.0) |
| ischemic stroke | | | | |
| Peripheral arterial disease | 1106 | (39.0) | 1150 | (39.9) |
| Coronary artery bypass | 871 | (30.7) | 881 | (30.6) |
| graft | | | | |
| surgery | | | | |
| Percutaneous coronary | 648 | (22.8) | 624 | (21.6) |
| Intervention | | | | |
| Hypertension* | 1225 | (43.2) | 1268 | (43.6) |
| Cholesterol, mg/dl† | 1882 | (85.8) | 1928 | (66.9) |
| Total >200.8 | | | | |
| High-density lipoprotein | 472 | (16.6) | 699 | (18.5) |
| 34.7 | | | | |
| Current smoking | 371 | (13.1 | 404 | (14.0) |
| B-Blockers | 1310 | (46.2) | 1348 | (48.8) |
| Lipid-lowering agents | 909 | (32.0) | 850 | (33.0) |
| Diuretics | 363 | (12.8) | 356 | (12.3) |
| Calcium-channel blockers | 1378 | (48.5) | 1427 | (49.5) |
| Left ventricular | 228 | (8.0) | 250 | (8.7) |
| hypertrophy | | | | |
| Microalbuminuria | 402 | (14.2) | 421 | (14.6) |

*History or blood pressure greater than 140/90 mm Hg.
†To convert total and high-density lipoprotein cholesterol from mg/dl to mmol/L, multiply by 0.0259

There were 102 individuals (3.6%) in the ramipril group compared with 155 (5.4%) in the placebo group (relative risk [RR], 0.66; 95% confidence interval [CI], 0.51-0.85; P<0.001) who reported a new diagnosis of diabetes. See FIG. 1. The proportion of patients diagnosed to have diabetes and a documented glycated hemoglobin of 110% or more above the upper limit of normal (1.8% vs. 3.0%; RR, 0.60; 95% CI, 0.43-0.85; P=0.003), those receiving an oral glucose lowering agent or insulin (2.1% vs. 3.6%; RR, 0.56; 95% CI, 0.41-0.77; P<0.001). Those with all criteria (1.3% vs. 2.5%; RR, 0.51; 95% CI, 0.34-0.76; P<0.001) were significantly lower in the ramipril group compared with the placebo group. Vitamin E and placebo did not differ in their effect on diabetes.

Because it is believed that ramipril reduced the risk of cardiovascular events and diabetic nephropathy, we assessed whether the higher occurrence of these clinical events in placebo-treated patients increased the likelihood of ascertainment of diabetes in this group. Similar stratified analyses by the occurrence of other outcomes was also examined. As noted in Table 2, the impact of ramipril on the development of diabetes could not be explained by any confounding factor such as preferential ascertainment in one group vs. the other or use of concomitant medications.

TABLE 2
Effect of Ramipril on the Development of Diabetes Using a Range of
Criteria and Stratified by the Occurrence of Specific Events*

| | | | | |
|---|---|---|---|---|
| Variables | Rampiril | Placebo | RR (95%) CI) | P Value |

New Diabetes†

| | | | | |
|---|---|---|---|---|
| With primary event | 9 (2.4) | 28 (5.5) | 0.48 (0.21-0.98) | .04 |
| No primary event | 93 (3.8) | 129 (5.4) | 0.69 (0.53-0.91) | .007 |
| With new MA or | 20 (5.6) | 98 (8.4) | 0.85 (0.38-1.12) | .12 |
| ON | | | | |
| No new MA or ON | 82 (9.3) | 119 (4.9) | 0.87 (0.51-0.89) | .005 |

New Diabetes With Glycated Hemoglobin ≧110%, ULN‡

| | | | | |
|---|---|---|---|---|
| With primary event | 7 (1.9) | 16 (3.4) | 0.59 (0.24-1.43) | .23 |
| No primary event | 45 (1.8) | 71 (3.0) | 0.81 (0.42-0.89) | .009 |
| With new MA or | 10 (2.8) | 25 95.8) | 0.47 (0.23-0.98) | .04 |
| ON | | | | |
| No new MA or ON | 42 (1.7) | 82 (2.6) | 0.66 (0.48-0.98) | .04 |

New Diabetes With Oral Agents or Insulin§

| | | | | |
|---|---|---|---|---|
| With primary event | 5 (1.3) | 16 (3.4) | 0.42 (0.15-1.14) | .08 |
| No primary event | 54 (2.2) | 89 (3.7) | 0.58 (0.42-0.82) | .002 |
| With new MA or | 14 (3.9) | 27 (6.3) | 0.61 (0.32-1.16) | .18 |
| ON | | | | |
| No new MA or ON | 45 (1.8) | 78 (3.2) | 0.56 (0.39-0.81) | .002 |

New Diabetes With Oral Agents or Insulin and Glycated
Hemoglobin ≧110%, ULN| |

| | | | | |
|---|---|---|---|---|
| With primary event | 4 (1.1) | 10 (2.1) | 0.64 (0.17-1.72) | .29 |
| No primary event | 32 (1.9) | 81 (2.5) | 0.61 (0.33-.78) | .001 |
| With new MA or | 6 (1.7) | 22 (5.1) | 0.32 (0.13-0.79) | .009 |
| ON | | | | |
| No new MA or ON | 30 (1.2) | 49 (2.0) | 0.80 (0.38-0.84) | .03 |

*RR indicates relative risk CI, confidence interval; MA, microalbuminuria; ON, overt nephropathy; ULN, upper limits of normal; and primary event, death, myocardial infarction, or stroke.
†Controlling for primary events and development of MA or ON, new diabetes with glycated hemoglobin 110% had a 0.67 RR (95% CI, 0..52-0.86).
‡Controlling for primary events and development of MA or ON, new diabetes with patient taking glucose-lowering therapy had a 0.58 RR (95% CI, 44-0.88).
§Controlling for primary events and development of MA or ON, new diabetes with patient taking glucose-lowering therapy had a 0.58 RR (95% CI, 42-0.79).
| |Controlling for primary events and development of MA or ON, new diabetes with patient taking glucose-lowering therapy had a 0.52 RR (95% CI, 35-0.78).

FIG. 2 demonstrates the results among subgroups of patients with different risk factors for developing diabetes. The results are consistent among those with a waist to hip ratio below or above the median of 0.93 or less or higher than 0.93 and consistent among those with a body mass index (BMI) of 27.7 or less or higher than 27.7, those with or without a history of hypertension, those receiving or not receiving β-blockers or diuretics at randomization. A higher proportion of individuals without diabetes who were randomized to the placebo group than those randomized to the ramipril group received diuretics or β-blockers (drugs that are associated with glucose intolerance or diabetes) during the study. However, the RR for diabetes in the subgroup of individuals who never took these drugs during the study was consistent with the overall results (RR, 0.62; 95% CI, 0.43-0.90).

In 4074 patients, weight was recorded at baseline and at study end. Weight increased by a mean (SD) of 0.98 (6.93) kg in the active group and 0.76 (8.10) kg in the control group.

These analyses indicate that ramipril reduced the risk of new diagnoses of diabetes among high risk individuals with no previous history of diabetes. The magnitude of the benefit appears to be large and, moreover, ACE-inhibitors are also believed to reduce macrovascular and microvascular complications of diabetes. Although the data on new diagnoses of diabetes were collected prospectively in the HOPE study, it was not a primary or secondary outcome of the trial. Therefore the results should be interpreted with caution. Nevertheless, the results are plausible given the clear statistical significance and consistency of results across subgroups, as well as using a range of approaches to diagnosing diabetes.

The Study to Evaluate Carotid Ultrasound changes in patients treated with Ramipril and vitamin E (SECURE) in which fasting glucose increased more with placebo (15.8 mg/dL [0.41 mmols]) than with ramipril (9.6 mg/dL (0.25 mmols]; P=0.03). Among the patients with diabetes in the HOPE study, there was a significant reduction in HbA_{1c} levels during serial annual recordings occurred during the first 2 years (absolute difference, 0.2%). In the UK Prospective Diabetes Study (UKPDS) and in the Captopril Prevention Project, patients randomized to receive ACE inhibitors had lower levels of HbA_{1c} or less development of diabetes compared with those taking β-blockers or diuretics. However, it is not clear whether the differences in development of diabetes observed in these studies are due to a protective effect of ACE inhibitors or an adverse effect of β-blockers or diuretics.

Hypokalemia substantially impairs the insulin secretory response to glucose, which may be favorably affected by ACE inhibitors. ACE inhibitors also lower aldosterone secretion and renal potassium wasting, which could preserve β-cell responsiveness. ACE inhibitors may increase islet blood flow and pancreatic β-cell perfusion by reducing angiotensin-2 mediated vasoconstriction in the [pancreas]. These effects may potentially slow or reverse the decline in β-cell function.

ACE inhibitors may reduce insulin resistance in skeletal muscles, increase insulin-mediated glucose disposal thereby decreasing the need for pancreatic insulin secretion. The increased insulin mediated glucose uptake by skeletal muscle in response to an ACE inhibitor is due to increased bradykinin-mediated nitric oxide production and not to reductions in angiotensin 2 production or action. Several observations suggest that agents that increase nitric oxide (such as ACE-inhibitors) may also increase insulin-mediated glucose uptake, which include that (1) both insulin-mediated vasodilation and skeletal muscle glucose metabolism are reduced in obese persons who do not have diabetes (i.e., individuals at risk for diabetes) and in individuals with type 2 diabetes, (2) inhibition of nitric oxide production reproduces this effect in lean individuals, and (3) the effect on insulin sensitivity is greater than can be accounted for by just increased skeletal muscle blood flow. ACE inhibitors may also reduce insulin resistance at the liver and fat cell, which would reduce hepatic glucose production and lower free fatty acid levels.

It is our belief that this data demonstrates that ramipril, an ACE inhibitor, reduces the risk of developing diabetes mellitus.

This Example is summarized as follows:

Design, Setting and Participants. The randomized, controlled Heart Outcomes Prevention Evaluation trial of 5720 patients older than 55 years without known diabetes but with vascular disease who were followed up for a mean of 4.5 years. The study included 267 hospitals in 19 countries and was conducted between 1994 and 1999.

Intervention. Patients were randomly assigned to receive ramipril, up to 10 mg/d (n=2837), or I

placebo (n=2883).

Main Outcome Measure. Diagnosis of diabetes determined from self-report at follow-up visits every 6 months, compared between the 2 groups.

Results. One hundred and two individuals (3.6%) in the ramipril group developed diabetes compared with 155 (5.4%) in the placebo group (relative risk [RR], 0.66-95% confidence interval [CI], 0.51-0.85, P<0.001). Similar results were noted when different diagnostic criteria were used; in the ramipril group, the RR for diagnosis of diabetes and hemoglobin A_{1c} greater than 110% was 0.60 (95% CI, 0.43-085), for initiation of glucose-lowering therapy, 0.56 (95% CI, 0.41-0.77), and for both, 0.51(95% CI, 0.34-0.76). These effects were also consistently seen in several subgroups examined.

Conclusions. It is our belief that angiotensin converting enzyme inhibitors, especially ramipril, are associated with lower rates of new diagnosis of diabetes in high-risk individuals.

In further support of the present invention, the following article, S. Yusuf et al.: JAMA, Ramipril and the Development of Diabetes, 286(15):1882-1885 (Oct. 17, 2001), is incorporated in its entirety herein by reference.

While the present invention has been described in the context of preferred embodiments and examples, it will be readily apparent to those skilled in the art that other modifications and variations can be made therein without departing from the spirit or scope of the present invention. Accordingly, it is not intended that the present invention be limited to the specifics of the foregoing description of the preferred embodiments and examples, but rather as being limited only by the scope of the invention as defined in the claims appended hereto.

Having described my invention, I claim:

1) a method of reducing diabetes in a patient who is at risk for developing diabetes, said method comprising:
administering to a patient, who is at risk for developing diabetes, an effective amount of an angiotensin converting enzyme inhibitor for sufficient period of time to prevent the development of diabetes in such patient.

2) A method according to claim 1, wherein the diabetes is Type 2 Diabetes.

3) A method according to claim 1, wherein the angiotensin converting enzyme inhibitor is ramipril.

4) A method according to claim 2, wherein the angiotensin converting enzyme inhibitor is ramipril.

5) A method according to claim 1, wherein β-cell function in such patient is slowed or reversed.

6) A method according to claim 1, wherein islet blood flow is increased.

7) A method according to claim 1, wherein pancreatic β-cell perfusion is increased.

8) A method according to claim 1, wherein insulin resistance in skeletal muscles is reduced.

9) A method according to claim 1, wherein insulin-mediated glucose disposal is increased.

10) A method according to claim 1, wherein insulin-mediated glucose uptake by skeletal muscles is increased.

11) A method according to claim 3, wherein the ramipril is administered in a dose of up to about 10 mg per day.

12) A method of slowing or reversing the decline of β-cell function in an individual comprising:
administering to an individual an effective amount of an angiotensin converting enzyme inhibitor for a sufficient period of time to prevent the decline of β-cell function in such individual.

13) A method according to claim 12, wherein the angiotensin converting enzyme inhibitor is ramipril.

14) A method of increasing islet blood flow in an individual comprising:
administering to an individual an effective amount of an angiotensin converting enzyme inhibitor for a sufficient period of time to increase islet blood flow in such individual.

15) A method according to claim 14, wherein the angiotensin converting enzyme inhibitor is ramipril.

16) A method of increasing pancreatic β-cell perfusion in an individual comprising:
administering to an individual an effective amount of an angiotensin converting enzyme inhibitor for a sufficient period of time to increase pancreatic β-cell perfusion in such individual.

17) A method according to claim 16; wherein the angiotensin converting enzyme inhibitor is ramipril.

18) A method of lowering aldosterone secretion and renal potassium wasting in an individual by comprising:
administering to an individual an effective amount of an angiotensin converting enzyme inhibitor for a sufficient period of time to lower aldosterone secretion and renal potassium wasting in such individual.

19) A method according to claim 18, wherein the angiotensin converting enzyme inhibitor is ramipril.
